(19) United States
(12) Patent Application Publication
(10) Pub. No.: US 2014/0199413 A1
(43) Pub. Date: Jul. 17, 2014

(54) MELATONIN AND AN ANTIMICROBIAL OR ANTIBACTERIAL AGENT FOR THE TREATMENT OF ACNE

(71) Applicant: Giorgio Endrici, Trento (TR) (IT)

(72) Inventor: Giorgio Endrici, Trento (TR) (IT)

(73) Assignee: NOVAERA S.R.L., Trento (TR) (IT)

(21) Appl. No.: 13/978,742

(22) PCT Filed: Jan. 5, 2012
(86) PCT No.: PCT/IB12/00013

§ 371 Date: Sep. 6, 2013

(30) Foreign Application Priority Data
Jan. 10, 2011 (IT) MI2011A000009

(51) Int. Cl. A61K 31/4045; A61K 31/155

(52) U.S. Cl. CPC A61K 31/4045; A61K 31/155
USPC 424/642; 514/415; 514/29; 514/24; 514/294; 514/154

(57) ABSTRACT

The invention relates to formulations for topical use comprising an antiseborrheic agent and an antimicrobial and/or antibacterial agent for the treatment of inflammatory dermatoses. In particular said formulations comprise melatonin and an antimicrobial or antibacterial agent, can be used in the pharmaceutical, cosmetic/cosmeceutical or dermatological field and are particularly suitable for the treatment of acne and of the clinical symptoms associated thereto.

### BACKGROUND ART

The present invention relates to a pharmaceutical, cosmetic/cosmeceutical or dermatological or base preparation of an antiseborrheic agent and an antimicrobial or antibacterial agent for the treatment of inflammatory dermatoses, in particular for the treatment of acne.

The term “acne” indicates a cutaneous disease, also defined as acne vulgaris. Acne is an inflammatory dermatosis involving the pilosebaceous unit, localized above all on the face and chest. The pathology is characterized by the presence, simultaneously or in subsequent stages, of comedones, papules, pustules or cysts, which at times can lead to scarring, chronic course and flareups.

When the pilosebaceous follicle becomes clogged, filling with sebum, epithelial cells and bacteria, this causes an open comedone, which represents the initial acne lesion.

The other lesions typical of the disease occur through subsequent inflammatory processes; these range from open comedones to small solid raised red areas (papules), to pus-filled elements (pustules), to deeper lesions, at times painful (nodes and cysts), to pus-filled elements (pustules).

Excessive production of sebum, known as seborrhea, is always present in acne. The occurrence of scarring depends on the severity, depth and duration of the cutaneous lesions. The causes of acne vulgaris are for the most part unknown. Some factors seem to play a fundamental role in its development, above all hyperkeratinization of the pilosebaceous duct, variations in the quality and quantity of sebum, actions of microbial flora, increase in androgen production and, last but not least, psychological factors. The disease can be worsened by the presence of Propionibacterium Acnes (P. acnes) the level of the infundibulum.

For this reason early and adequate treatment of acne is very important, as it is a particularly distressing condition, above all at psychological and emotional level.

Notwithstanding the high incidence of the disease in the world population, no single therapeutic agent capable of improving all the factors involved in the etiopathogenesis of the disease is yet available. Topical therapy is often preferred for its safety in relation to other types of treatment. Topical therapies currently available include comedolytic, antibiotic, antimicrobial and anti-inflammatory agents.

There is the need to identify active ingredients for topical use which overcome the limits of prior art and which are therefore safe, while having a particularly high action potential and efficacy.

Melatonin (N-[2-(5-methoxy-1H-indol-3-yl)ethyl]ethanamide) is a hormone with multiple bioactive properties in humans which is well known and has been described in the literature for some time. Melatonin is produced by the pineal gland and stimulated by beta-adrenergic receptors. The melatonin levels in serum exhibit a circadian rhythm, low during the day, increasing in the evening and at maximum levels during the night. Melatonin participates in regulating numerous physiological processes such as seasonal biological rhythms, daytime sleepiness, aging and modulation of immunological defence reactions. Moreover, melatonin has a highly lipophilic molecular structure which facilitates membrane penetration and also acts to eliminate intra- and extra cellular free radicals. Besides its anti-radical and anti-oxidant activity, a possible role of this hormone in the etiology of some conditions such as psoriasis, eczema and malignant melanoma, has been hypothesized.

Due to the possible involvement of melatonin in other physiological functions, besides sleep-wake rhythm, the hormone has also been used in the treatment of some conditions. For example, US2008/0131496 describes the use of melatonin encapsulated in liposomes to treat itching and irritation of the skin, for example caused by sunburn, by insect bites, or by acne. More in general, the aforesaid document describes the use of melatonin encapsulated in liposomes to treat itching deriving from the release of hystamine and activation of an allergic immunological response.

The above described results suggest that melatonin can have therapeutic effects in the treatment of itching in cutaneous conditions deriving from the release of hystamine and activation of an allergic immunological response.

The patent application filed with No. TN2005A0013 describes the possible involvement of melatonin in the translocation of androgen receptors and its use as inactivator of dihydrotestosterone (DHT). TN2005A0013 describes the possible use of melatonin to treat acne, in concentration ranges from 0.01% to 0.0099% and from 0.011% to 1%.

Therefore, there is the real need to identify an effective therapy that can be implemented from a practical viewpoint, in the treatment of inflammatory dermatoses, in particular acne. The multifactorial components of this disease oblige patients to undergo long and particularly costly treatments.

Therefore, there is the real need to find additional formulations with high bioavailability and high compliance for the patient suffering from inflammatory dermatosis, in particular acne, which can be used for a relatively lung period of time without serious contraindications and with increased pharmacological activity promote the cure and eradication of the disease.

### OBJECTS OF THE INVENTION

The object of the present invention is to provide a composition, in particular a pharmaceutical, cosmetic/cosmeceutical or dermatological composition to use for the treatment of cutaneous conditions, the latter being characterized by inflammatory dermatosis.

Another object of the present invention is to provide a pharmaceutical, cosmetic/cosmeceutical or dermatological composition for the treatment of cutaneous conditions such as acne and the clinical symptoms associated thereto.

Yet another object of the invention is to provide a composition which can have a cosmetic/cosmeceutical and pharmaceutical use.

A further object of the present invention is to provide a composition which is effective both for topical use in the treatment of acne and which, at the same time, does not leave the skin oily and allows the application of cosmetics and/or sun protection cream without damages or negative interactions.

### DESCRIPTION OF THE INVENTION

These and yet other objects and related advantages which shall be more apparent from the description below are accomplished by a composition comprising melatonin and at least one antimicrobial and/or antibacterial agent, besides the usual excipients and additives.

In particular, in the composition according to the invention, melatonin performs an action as antiseborrheic agent in synergic association with said at least one antimicrobial and/or antibacterial agent.

The composition according to the present invention overcomes the limits of prior art due to the surprising synergic action observed between melatonin and said antimicrobial and/or antibacterial agent in the treatment of cutaneous conditions such as inflammatory dermatosis.

Therefore, the subject matter of the present invention is also a composition comprising melatonin and at least one antimicrobial and/or antibacterial agent for use in the treatment of inflammatory dermatosis.

More in particular, the composition forming the subject matter of the invention is advantageously used for the treatment of acne.

The subject matter of the present invention is also a composition comprising melatonin and at least one keratolytic agent, and also its use in the treatment of inflammatory dermatosis.

Patients suffering from acne, regardless of the degree of severity of the condition from which they are suffering, must use more than one formulation for topical use, often even several times a day. Regardless of the gender of the patient suffering from acne, the use of formulations for topical use in which the active ingredient is contained in an oil-based formulation makes the face, the part of the body most affected by acne and also the part most exposed, oily. This condition creates considerably psychological distress in the patient, as he or she is obliged to deal with interpersonal relationships in a state of inadequacy. The problems and difficulties in interpersonal relationships are even greater and more evident when the subjects suffering from acne are female. In fact, the psychological malaise is even greater, as the disease often makes it impossible to apply cosmetic products. Cosmetic products on the market, such as foundation and the like, can further clog the pilosebaceous pores and worsen the state of the disease. For patients suffering from acne, even suncreams, which are recommended by physicians and dermatologists to protect the skin against harmful sun rays, can represent a threat for worsening of the disease.

The present invention overcomes the limits of prior art, as it improves the quality of life of the patient suffering from acne.

The composition according to the present invention solves the aforesaid problems caused by the topical formulations currently available.

The composition can be used in the pharmaceutical or cosmetic/cosmeceutic field and it is intended in particular for topical use.

The compositions or formulations according to the present invention therefore comprise melatonin, as antiseborrheic agent associated to other active ingredients with synergic action, such as antimicrobial or antibacterial agents, in the treatment and prevention of acne.

Said additional antimicrobial or antibacterial active ingredients are selected from antimicrobial agents such as chlorhexidine, benzoyl peroxide, pharmaceutically acceptable forms of zinc salts such as zinc oxide, or antibacterial agents such as azelaic acid, those belonging to the class of macrolides such as erythromycin and derivatives thereof, those belonging to the class of tetracyclines such as tetracycline and derivatives thereof, the class of lincosamides such as clindamycin and derivatives thereof, the class of fluoroquinolones such as nadifloxacin and derivatives thereof, natural or synthetic substances with inhibitory activity against P. acnes such as 1-pentadecanol and derivatives thereof, cedrene, caryophyllene, longifolene.

The subject matter of the present invention is also a pharmaceutical formulation or composition based on an antiseborrheic agent such as melatonin and at least one antimicrobial and/or antibacterial agent optionally with the addition of pharmaceutically acceptable salts and/or additives.

The subject matter of the present invention is also a pharmaceutical formulation or composition based on an antiseborrheic agent such as melatonin and at least one keratolytic agent optionally with the addition of pharmaceutically acceptable salts and/or additives.

The term “pharmaceutical formulation or composition” are intended as compositions or formulations comprising melatonin, and at least one antimicrobial and/or antibacterial agent in addition to other components and/or additives compatible with the pharmaceutical practice.

The term “addition of pharmaceutically acceptable salts” is intended here as referring to all those salts that from a biological, preparation and formulation point of view are compatible with the pharmaceutical practice.

According to the present invention, melatonin can be incorporated in a variety of formulations suitable for topical release of the active ingredients. Topical formulations suitable for the treatment and prevention of inflammatory dermatosis, in particular of acne, are creams, lotions, mousses, sprays, emulsions, gels and the like, compatible with the preparation according to methods commonly known in the state of the art.

The compositions according to the invention can comprise the pharmaceutically acceptable known excipients, such as carriers, preserving agents, surfactants, thickening agents, scents, chelating agents, water, alcohols, anti-oxidants, antiseptics, colourants and UV absorbers.

In particular, the composition forming the subject matter of the invention is advantageously used for the preparation of a medication for the treatment of acne.

The formulations or compositions according to the invention are suitable for topical administration of the active ingredients.

In a preferred form thereof, the formulation according to the invention therefore comprises as essential active substance melatonin together with an antimicrobial agent selected from: chlorhexidine, benzoyl peroxide, zinc salts and or antibacterial agent selected from: azelaic acid, macrolides such as erythromycin and derivatives thereof, tetracyclines such as tetracycline and derivatives thereof, lincosamides such as clindamycin and derivatives thereof, fluoroquinolones such as nadifloxacin and derivatives thereof, natural or synthetic substances with inhibitory activity against P. acnes such as 1-pentadecanol and derivatives thereof, cedrene, caryophyllene, longifolone.

The compositions or formulations according to the invention contain a quantity of melatonin between 0.001% and 5% by weight with respect to the total weight of the formulation and a quantity of antibacterial and/or antimicrobial agent greater than 0.05% and less than 25%, again with respect to the total weight of the formulation.

According to a preferred aspect of the invention, the compositions or formulations contain a quantity of melatonin between 0.001% and 5%, preferably between 0.001% and 2%, even more preferably between 0.001% and 1%, preferably between 0.001% and 0.01% or between 0.01% and 1%, the concentration of 0.005% being particularly preferred, each of the aforesaid percentages being expressed by weight with respect to the total weight of the composition.

According to another preferred aspect of the invention, the compositions or formulations contain a quantity of melatonin between 2% and 5%, preferably between 2% and 2.5%, even more preferably between 1% and 2%, the concentration of 2.3% being particularly preferred, each of the aforesaid percentages being expressed by weight with respect to the total weight of the composition.

Preferably, the compositions or formulations according to the invention, in addition to the quantity of melatonin, contain a quantity of chlorhexidine in a concentration range from 0.05% to 1%, preferably from 0.05% to 0.1%, and even more preferably from 0.05% to 0.07%, quantities expressed by weight % with respect to the total weight of the composition.

Even more preferably, the composition or formulation according to the invention contains a quantity of melatonin of 0.005% by weight and a quantity of chlorhexidine of 1% by weight with respect to the total weight of the composition.

Moreover, according to another aspect thereof, one of the preferred compositions or formulations according to the invention contains a quantity of melatonin of 0.005% by weight and a quantity of chlorhexidine of 0.1% by weight with respect to the total weight of the composition.

Moreover, according to another preferred aspect thereof one of the compositions or formulations according to the invention contains a quantity of melatonin of 2.3% by weight and a quantity of chlorhexidine of 0.1% by weight with respect to the total weight of the composition.

Preferably, in the formulation according to the invention, the melatonin is present in a concentration of 0.005% by weight with respect to the total weight of the composition.

The formulations or compositions according to the present invention comprise as active ingredient melatonin associated respectively to erythromycin from 1% to 5%, and/or benzoyl peroxide from 7% to 15%, and/or clindamycin from 0.05% to 4%, and/or azelaic acid from 15% to 25%, and/or nadifloxacin from 0.5% to 5%, and/or zinc salts such as zinc oxide from 5% to 20%, and/or meclocycline from 0.5% to 5%, each of the aforesaid percentages being expressed by weight with respect to the total weight of the composition.

Preferably, the formulations or compositions according to the present invention comprise, as active ingredient, melatonin associated respectively to erythromycin at 3%, and/or benzoyl peroxide 10%, and/or clindamycin 1%, and/or azelaic acid 20%, and/or nadifloxacin 1%, and/or zinc oxide 10%, and/o meclocycline 1%, each of the aforesaid percentages being expressed by weight with respect to the total weight of the composition.

According to a preferred aspect, the formulations or compositions of the present invention comprise melatonin and at least one antimicrobial and at least one keratolytic agent selected from retinoids, salicylic acid, benzoyl peroxide.

According to another aspect thereof, the formulations or compositions of the present invention comprise melatonin and at least one antimicrobial agent.

According to yet another aspect thereof, the formulations or compositions of the present invention comprise melatonin and at least one antibacterial agent.

The subject matter of the present invention is therefore a pharmaceutical composition with antiseborrheic, antimicrobial and antibacterial synergic action for the treatment of inflammatory dermatosis, characterized by excellent pharmacokinetic and bioavailability parameters.

The composition can be used, for example, in the treatment of acne, Said composition ensures an effective decrease in the production of sebum and in cutaneous infections correlated to excessive accumulation thereof and which may be complicated and sustained by bacterial agents, in particular due to the surprising synergic action of melatonin associated to the antibacterial and/or antimicrobial agent.

In another embodiment thereof, the formulation according to the invention therefore comprises, as essential active ingredient, melatonin together with a keratolytic agent.

Due to its surprising characteristics, the composition according to the invention allows a regression of acne also in particular conditions, for example in case of acne defined as clinically severe.

The use of the composition according to the invention is also particularly advantageous to reduce pustules.

The formulation for topical use according to the invention can also be used several times a day, preferably twice a day, spaced apart over a period of 24 hours.

All the concentrations indicated in the present application are considered as percentages by weight of each of the active ingredients with respect to the total weight of the formulation/composition.

The high selectivity and the high bioavailability of the composition forming the subject matter of the invention, ensure that it can be used advantageously for the treatment of inflammatory dermatoses, particular of acne.

As indicated above, in acne the inflammatory process of the hair follicle and of the associated sebaceous gland are particularly evaluated. In particular, characterization of the condition is implemented on the basis of the presence of papules and pustules deriving from the original lesion: the comedone. In this way three stages of acne are distinguished: mild, moderate and severe, divided according to severity on the basis of the presence and of the approximate number of comedones and lesions, with or without inflammation. Details are provided in the Table 1 below:

TABLE 1

| | | Lesions with | |
|---|---|---|---|
| | | inflammation | |
| Severity of acne | Comedones | (papules/pustules) | Other Lesions |
| Mild | Less than 20 | Less than 15 | Less than 30 |
| Moderate | 20-100 | 15-50 | 30-125 |
| Severe | More than 100 | More than 50 | More than 125 |

In order to determine the efficacy of the formulation farming the subject matter of the present invention when used in the treatment of inflammatory dermatoses, in particular acne, the studies and experiments set forth below were conducted.

40 patients suffering from mild acne, identified on the basis of Table 1, were divided into four groups and treated or 4 weeks, twice a day, with topical applications respectively of placebo formulation (Control Group), formulation comprising 0.005% of melatonin (Test Group 2) according to the state of the art, formulation comprising 0.1% of chlorhexidine (Test Group 1) according to the state of the art or with a formulation comprising 0.005% of melatonin and chlorhexidine 0.1% (Test Group 3) according to the present invention. The course of the acne was evaluated in each group in terms of regression of pustules, reduction in the diameter and presence of lesions with or without inflammation, reduction in the production of sebum.

| | CONTROL | TEST | TEST | TEST |
|---|---|---|---|---|
| Weeks of | GROUP | GROUP 1 | GROUP 2 | GROUP 3 |
| treatment | (10 pat.) | (10 pat.) | (10 pat.) | (10 pat.) |
| 2 | 0 | 1 | 1 | 3 |
| 4 | 1 | 1 | 2 | 4/5 |

0: no improvement,
1: mild improvement
2: modest improvement
3: moderate improvement,
4: significant improvement,
5: complete healing.

The results indicated in Table 2 allow the efficacy of the treatment obtained through the use of the formulation of the invention to be established. In particular, it can be noted that after the second week of treatment, the patients of Group 3 exhibit an improvement evaluated by the experimenter as significant. In fact, from the second week significant reductions are noted in lesions in general, including inflammatory lesions, in the production of sebum and regression of pustules. After four weeks of treatment, complete healing was observed in 20% of cases. The patients treated state that they were already satisfied from the second week of treatment and remain compliant with the study. These results show that the formulation of the invention comprising 0.005% of melatonin and chlorhexidine 0.1% has excellent efficacy and tolerability and confirms the continuous reduction of cutaneous symptoms typical of acne and high satisfaction of the treated patient.

40 patients suffering from moderate acne, identified on the basis of Table 1, were divided into three groups and treated for 8 weeks, twice a day, with topical applications: respectively of placebo formulation (Control Group), formulation comprising 0.005% of melatonin (Test Group 2) according, to the state of the art, formulation comprising 0.1% chlorhexidine (Test Group 1) according to the state of the art or with a formulation comprising 0.005% of melatonin and chlorhexidine 0.1% (Test Group 3) according to the present invention. The course of the acne was evaluated in each group in terms of regression of pustules, reduction in the diameter and presence of lesions with or without inflammation, reduction in the production of sebum.

| Weeks of | CONTROL | TEST | TEST | TEST |
|---|---|---|---|---|
| treatment | GROUP | GROUP 1 | GROUP 2 | GROUP 3 |
| 2 | 0 | 0 | 1 | 2 |
| 4 | 0 | 1 | 1 | 3 |
| 6 | 1 | 1 | 2 | 4 |
| 8 | 1 | 1 | 2 | 4/5 |

0: no improvement,
1: mild improvement
2: modest improvement
3: moderate improvement,
4: significant improvement,
5: complete healing.

The results indicated in Table 3 allow the efficacy of the treatment obtained through the use of the formulation of the invention to be established. In particular, it can be noted that after the second week of treatment, the patients of Group 3 exhibit an improvement evaluated by the experimenter as significant. From the fourth week of treatment, significant reductions are noted both in inflammatory and non-inflammatory lesions, in the production of sebum and regression of pustules. After eight weeks of treatment, complete healing was observed in 20% of eases. The patients treated state that they were already satisfied from the second week of treatment and remain compliant with the study. These results show that the formulation of the invention comprising 0.005% of melatonin and chlorhexidine 0.1% has excellent efficacy and tolerability and confirms the continuous reduction of cutaneous symptoms typical of acne and high satisfaction of the patient treated.

40 patients suffering from severe acne, identified on the basis of Table 1, were divided into three groups and treated for 12 weeks, twice a day, with topical applications respectively of placebo formulation (Control Group), formulation comprising 0.005% of melatonin (Test Group 2) according to the state of the art, formulation comprising 0.1% chlorhexidine (Test Group 1) according to the state of the art or with a formulation comprising 0.005% of melatonin and chlorhexidine 0.1% (Test Group 3) according to the present invention. The course of the acne was evaluated in each group in terms of regression of pustules, reduction in the diameter and in the presence of lesions with or without inflammation, reduction in the production of sebum.

| Weeks of | CONTROL | TEST | TEST | TEST |
|---|---|---|---|---|
| treatment | GROUP | GROUP 1 | CROUP 2 | GROUP 3 |
| 2 | 0 | 0 | 0 | 1 |
| 4 | 1 | 1 | 1 | 2 |
| 6 | 1 | 0 | 1 | 3 |
| 8 | 0 | 1 | 1/2 | 3/4 |
| 10 | 0 | 1 | 1/2 | 4 |
| 12 | 0 | 1/2 | 2 | 4/5 |

0: no improvement,
1: mild improvement
2: modest improvement
3: moderate improvement,
4: significant improvement,
5: complete healing.

The results indicated in Table 4 allow the efficacy of the treatment obtained through the use of the formulation of the invention to be established. In particular, it can be noted that after the fourth week of treatment, the patients of Group 3 exhibit an improvement evaluated by the experimenter as significant. After the sixth week of treatment significant reductions are noted in inflammatory and non-inflammatory lesions, in the production of sebum and regression of pustules. After eleven and twelve weeks of treatment, complete healing was observed in 20% of cases. The patients treated state that they were already satisfied from the second week of treatment and remain compliant with the study. These results show that the formulation of the invention comprising 0.005% of melatonin and chlorhexidine 0.1% has excellent efficacy and tolerability and confirms the continuous reduction of cutaneous symptoms typical of acne and high satisfaction of the patient treated.

According to the indications above for the three categories of patients it can be asserted that the formulation according to the invention is efficacious in the treatment of acne, it is capable of reducing the clinical symptoms associated to acne, it is suitable for topical treatment in the short, medium or long term showing continuous and constant improvement and at the same time reducing side effects, such as irritation and dryness of the treated skin.

The present invention is better illustrated through the examples, set forth which are not to be considered in any way limiting.

### EXAMPLE 1

### Formulation in Cream

The formulation according to the present invention comprises:
melatonin 0.005%
chlorhexidine 1%
excipients and water to 100%.

### EXAMPLE 2

### Formulation in Cream

The formulation according to the present invention comprises:
melatonin 0.00:5%
chlorhexidine 0.1%
excipients and water to 100%.

1. Composition comprising melatonin and at least one antimicrobial and/or antibacterial agent.

2. Composition according to claim 1, wherein said antimicrobial agent is selected from the group consisting of chlorhexidine, benzoyl peroxide, and zinc salts and said antibacterial agent is selected from the group consisting of the compounds belonging to the classes of macrolides, azelaic acid, tetracyclines, lincosamides, fluoroquinolones, and natural or synthetic substances with inhibitory activity against P. acnes.

3. Composition according to claim 2, wherein said compounds belonging to the class of macrolides are selected from: erythromycin and derivatives thereof, said compounds belonging to the class of tetracyclines are selected from: tetracycline and derivatives thereof, said compounds belonging to the class of lincosamides are selected from: clindamycin and derivatives thereof, said compounds belonging to the class of fluoroquinolones are selected from: nadifloxacin and derivatives thereof, said compounds belonging to the class of natural or synthetic substances with inhibitory activity against P. acnes are selected from: 1-pentadecanol and derivatives thereof, cedrene, caryophyllene, longifolene.

4. Composition according to claim 1, wherein melatonin is at a concentration within 0.001% and 5%, if needed said antimicrobial agent is at a concentration within 0.05% and 4%, if needed said antibacterial agent is at a concentration within 0.05% and 4%, by weight with respect to the total weight of the composition.

5. Composition according to claim 1, wherein it comprises melatonin and chlorhexidine.

6. Composition according to claim 5, wherein said melatonin is present at a concentration of 0.005% and said chlorhexidine is present at a concentration of 1% by weight with respect to the total weight of the composition.

7. Composition according to claim 5, wherein said melatonin is present at a concentration of 0.005% and said chlorhexidine is present at a concentration of 0.1% by weight with respect to the total weight of the composition.

8. Composition according to claim 4, wherein said melatonin is associated to erythromycin present in concentration ranges from 1% to 5%, benzoyl peroxide present in concentration ranges from 7% to 15%, clindamycin present in concentration ranges from 0.05% to 4%, azelaic acid present in concentration ranges from 15% to 25%, nadifloxacin present in concentration ranges from 0.5% to 5%, zinc oxide present in concentration ranges from 5% to 20%, meclocycline present in concentration ranges from 0.5% to 5%, by weight with respect to the total weight of the composition.

9. Pharmaceutical compositions comprising the formulation according to claim 1, in addition to other components and/or additives compatible with the pharmaceutical practice.

10. Composition according to claim 1 for use in the topical treatment of diseases characterized by inflammatory dermatosis.

11. Composition according to claim 1 for use in the topical treatment of acne.

12. A method for the topical treatment of diseases characterized by inflammatory dermatosis, comprising topically applying a composition according to claim 1, to an area of a patient in need thereof.

13. The method of claim 12, wherein the disease is acne.
